(19) United States
(12) Patent Application Publication
(10) Pub. No.: US 2011/0209716 A1
(43) Pub. Date: Sep. 1, 2011

(54) BABY RESTRAINING DEVICE

(76) Inventor: Adrian Scarsbrook, Bickley (GB)

(21) Appl. No.: 12/672,787

(22) PCT Filed: Aug. 19, 2008
(86) PCT No.: PCT/GB2008/002794

§ 371 Date: Feb. 9, 2010

(30) Foreign Application Priority Data
Aug. 20, 2007 (GB) 0716214.2

(51) Int. Cl. A61F 5/37

(52) U.S. Cl. 128/872

(57) ABSTRACT

The kit of parts for forming the device includes a first base member (12) having securing means (20) for securing it to the upper surface (24) of a baby nappy changing mat (10) at a first location (26) and engagement means (28) formed on its upper surface (24) for releasably engaging at least two upper arm restraint members (16) selectably in a plurality of laterally disposed positions. At least two upper arm restraint members (16) are provided, each in the form of a loop of adjustable circumference and each having, on its outer surface, engagement means (32) for releasably engaging the upper arm restraint member (16) with the first base member (12). A second base member (14) has securing means (42) for securing it to the upper surface (24) of a changing mat (10) at a second location (46) spaced from the first location (26) and engagement means (48) formed on its upper surface (50) for releasably engaging a torso restraint member (18). A torso restraint member (18) is in the form of a loop of adjustable circumference having, on its outer surface (54), engagement means (52) for releasably engaging the torso restraint member (18) with the second base member (14). The arrangement can conveniently be used with an existing changing mat and is fully adaptable to the size and shape of the baby.

### FIELD OF THE INVENTION

The present invention relates to a baby restraining device for use with a baby nappy (i.e. diaper) changing mat and in particular to a kit of parts for forming such a device.

### BACKGROUND OF THE INVENTION

It is common practice to change a baby's nappy on a changing mat, which typically comprises a flat upholstered surface, usually formed of a plastics material, having a raised perimeter to reduce the risk of the baby rolling off the mat and contain any fluids.

Some babies can be particularly mobile, making it difficult for the nappy changing to take place, especially for a clean nappy to put on to the baby in a properly aligned manner and additionally raising safety issues. The parent or guardian undertaking the exercise has to perform certain functions with two hands, while at least one of those hands is required to restrain the baby's movements. Furthermore, when the baby's hands are able to reach below waist level during the nappy changing process, further hygiene issues arise from the need to ensure that the baby's hands are kept clean.

A number of proposals have been made in the art to address these problems. For example, United States patent application US2005/0236002 (Cooley) describes a diaper changing restraint vest for restraining the upper torso of a child or infant while their diaper is being changed. The restraint provides two chest straps and two shoulder straps which initiate from behind the child. It attaches to, and is meant to be used in conjunction with, a stiff unbendable changing pad or table. It can be removed from the changing pad and easily transported to be used on public changing stations. The restraint keeps a child from rolling over or escaping while its diaper is being changed.

While the device proposed by Cooley could be used with an existing changing mat, it suffers from reduced adaptability. Babies come in a variety of shapes and sizes and grow rapidly. A restraining device therefore needs to have adaptability not only to accommodate differences in the circumference of the torso and the arms but also differences in the width of the shoulders and the length of the body. The Cooley device does not offer this adaptability. Furthermore, Cooley does not address the hygiene issue referred to above.

German Offenlegungschrift DE 4128255 (Neumann) describes a nappy-changing base intended to keep the baby safe from slipping or falling which consists of a padded sleeve having an insertion gap into which is placed a stabilising surface on which are fixture points. Holder strips, on which are hooked strips, are threaded through guides. The top ends of the holder strips have a padded strip. Part of the holder strips below the stabilising surface possesses a padded strip as well as a hooked strip. United States design patent US D 393561 (Hayes) shows a similar construction. These devices suffer from the disadvantage that the changing mat has to be modified to accommodate them, whereas it would certainly be preferable to have an arrangement which can be used with an existing changing mat. Furthermore, neither Neumann nor Hayes addresses the hygiene issue referred to above.

I am also aware of British patent GB 2357965 (Rabbitts) which discloses a baby changing mat which has a rigid base supporting shoulder and waist straps to anchor the upper torso of the baby to the mat. The mat is provided with heel recesses for receiving the heels of a carer seated on the floor. When the carer kneels on the floor, the knees can engage knee recesses. A raised peripheral wall provides a barrier to fluids gravitating towards the carer. Attaching strips enable an absorbent cloth to be fastened to the mat in a position to receive the lower torso of the baby. The cloth can be lifted at one end to provide a barrier between the baby and the carer.

Although not directed to a baby nappy changing device, I am also aware of International patent publication WO 94/13177 (Bowman) which describes a medical patient restraint device for restraining patients during surgical procedures such as the circumcision of infants. The restraint device has a planar surface which is sized to receive the patient in a lying position. A pair of support straps are fixed to a support member and extend along the side edges of the restraint device in a parallel relationship. The support straps are spaced so as to receive the patient therebetween. Individual restraining straps are provided for releasable connection to the respective wrists and legs of the patient. Mating fasteners are fixed to the support straps and the restraining straps so that the restraining straps may be releasably attached to the support straps so as to permit the patient to be restrained without the restraining straps extending across the patient's torso. Such a device is unsuitable for use as a baby nappy changing device.

It is thus an objective of the present invention to overcome the disadvantages of the prior art and in particular to provide an arrangement which can conveniently be used with an existing changing mat and which is fully adaptable to the size and shape of the baby.

### SUMMARY OF THE INVENTION

According to the invention there is provided a kit of parts for forming a baby restraining device for use with a baby nappy changing mat, the kit comprising:

(i) a first base member having securing means for securing it to the upper surface of a baby nappy changing mat at a first location and engagement means formed on its upper surface for releasably engaging at least two upper arm restraint members selectably in a plurality of laterally disposed positions;

(ii) at least two upper arm restraint members each in the form of a loop of adjustable circumference and each having, on its outer surface, means for releasably engaging the restraint member with the first base member;

(iii) a second base member having securing means for securing it to the upper surface of a changing mat at a second location spaced from the first location and engagement means formed on its upper surface for releasably engaging a torso restraint member; and

(iv) a torso restraint member in the form of a loop of adjustable circumference having, on its outer surface, engagement means for releasably engaging the torso restraint member with the second base member.

The baby restraining device formed from the kit according to the invention holds the baby safely in position whilst being changed, preventing the baby from twisting and/or crawling away. It allows the baby to be held more snugly and can be used in such a manner as to prevent the baby's hands from reaching below waist level during nappy changing and can be altered to allow for baby's growth. The kit has the flexibility of being usable with a wide range of changing mats. It has been found that use of the baby restraining device formed from the kit according to the invention has a calming influence on the baby in that the baby becomes more. passive.

The first base member is suitably in the form of an elongate band, although other shapes may be adopted. The first base member is preferably formed in one piece, although a two-piece base member could be used, each piece having securing means for securing it to the upper surface of a baby nappy changing mat at left and right first locations and each piece having engagement means formed on its upper surface for releasably engaging respectively one or another of the upper arm restraint members selectably in laterally disposed positions.

The second base member is preferably non-integral with the first base member, although it is possible for a single component to represent both the first and second base members, provided that such a component, when secured to the changing mat, has a lateral portion which represents and first base member and a longitudinal portion, extending away from the lateral portion to represent the second base member.

The securing means of the first and/or second base members are preferably formed on their lower surfaces, so that the members may easily be secured to the changing mat. The securing means is preferably in the form of tacky adhesive having sufficient adhesive strength to the surface of the mat so as not to be dislodged in the event of the baby wriggling.

The engagement means of the first base member, and/or of the second base member, may be in the form of one element of a hook-and-loop system, such as a Velcro™ system (Velcro Industries B.V.), while the engagement means of each of the upper arm restraint members and/or the torso restraining member respectively is in the form of the other element of that system. Other releasable engagement means could alternatively be used, such as other physical, magnetic or electrostatic devices. However, it is preferred that the engagement means of the first base member extends substantially continuously in the lateral direction across the upper surface of the first base member, thereby enabling the spacing between the loops of the upper arm restraint members to be adjusted for best comfort for the baby. Similarly, the engagement means of the second base member preferably extends substantially continuously at least in a longitudinal direction along the upper surface of the second base member, so as to provide for a maximum range of adjustment as the baby grows.

In a most preferred embodiment, particularly in the case of the engagement of the upper arm restraining members with the first base member, the engagement means are such as to provide orientation-independent engagement between the first base member and the upper arm restraint members, so that the loops of the upper arm restraint members may lie in planes which are not necessarily parallel to each other, e.g. planes which converge towards each other in the direction away from the baby's head.

The upper arm restraint members are each suitably in the form of a strap, which may be padded for additional comfort. The engagement means of each upper arm restraint member is preferably located midway along the strap and co-operating means are provided towards the free ends of the strap to enable the strap to be formed into a loop with adjustable circumference. These co-operating means are, for example, in the form of a hook-and-loop system with one element of the system being located on the outside of the strap towards one free end thereof while the other element of the system is located on the inside of the strap towards the other free end thereof.

For additional ease of use it is possible to provide the elements of the hook-and-loop system both on the inside and the outside towards both free ends of each upper arm restraint strap.

The torso restraint member is ideally in the form of a strap, which may be padded for extra comfort. The engagement means of the torso restraint member may be located midway along the strap with co-operating means being provided towards the free ends of the strap to enable the strap to be formed into a loop with adjustable circumference. These co-operating means may be in the form of a hook-and-loop system with one element of the system being located on the outside of the strap towards one free end thereof while the other element of the system is located on the inside of the strap towards the other free end thereof. For additional ease of use it is possible to provide the elements of the hook-and-loop system both on the inside and the outside towards both free ends of the torso strap.

The kit may further include a stiffening bar, to be secured to the lower surface of the changing mat, at approximately shoulder level, to further restrain movement of larger and more active babies, especially where the changing mat is formed of more flexible material.

The lower surface of the changing mat may be provided with means, such as one or more self adhesive pads to secure the mat to a changing table.

The invention also provides a baby restraining device comprising:

(i) a baby nappy changing mat;

(ii) a first base member having securing means securing it to the upper surface of the baby nappy changing mat at a first location and engagement means formed on its upper surface for releasably engaging at least two upper arm restraint members selectably in a plurality of laterally disposed positions;

(iii) at least two upper arm restraint members each in the form of a loop of adjustable circumference and each having, on its outer surface, engagement means for releasably engaging the upper arm restraint member with the first base member;

(iv) a second base member having securing means securing it to the upper surface of the changing mat at a second location spaced from the first location and engagement means formed on its upper surface for releasably engaging a torso restraint member; and

(v) a torso restraint member in the form of a loop of adjustable circumference having, on its outer surface, engagement means for releasably engaging the torso restraint member with the second base member.

The invention will now be further described, purely by way of example, with reference to the accompanying drawings, in which:

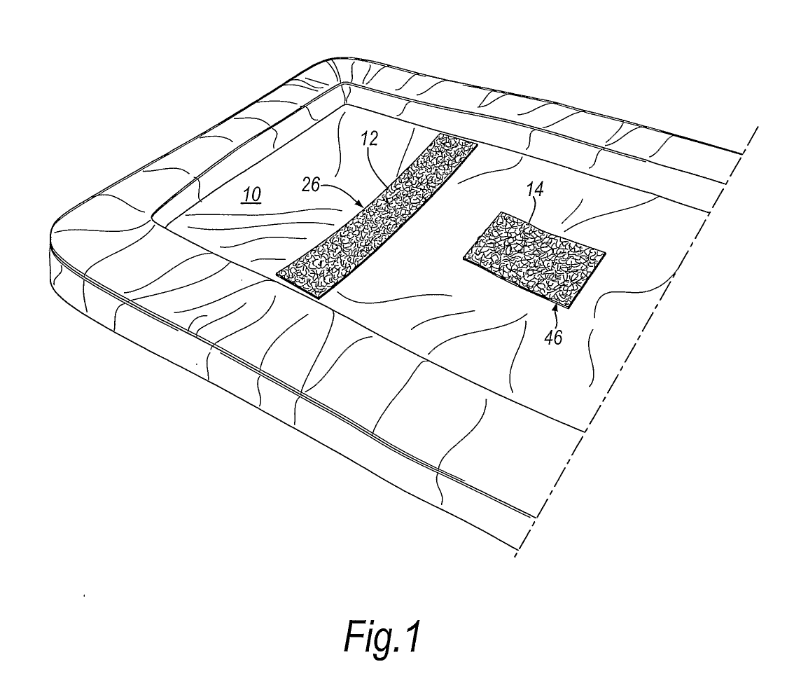

FIG. 1 shows the base members of a kit according to the invention, secured to a changing mat;

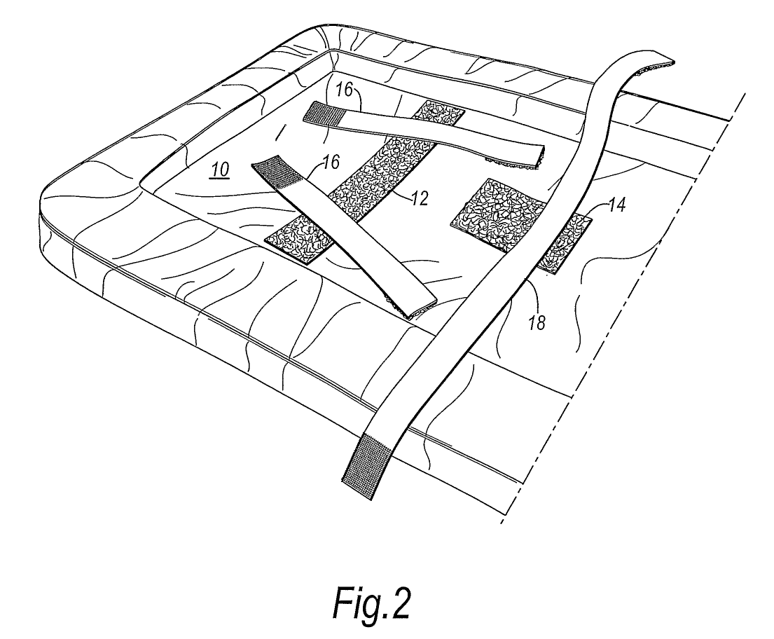

FIG. 2 shows the upper arm restraints and torso restraint of the kit engaged with the base members;

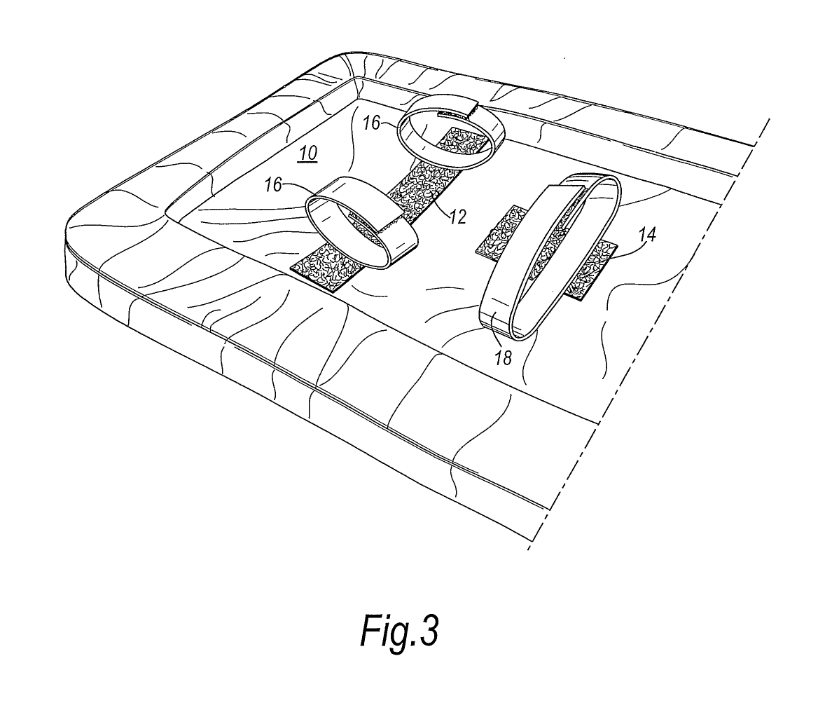

FIG. 3 shows the upper arm restraints and torso restraint of the kit in their looped configuration;

FIG. 4 is a cross-sectional view of the first base member of the kit as it is being secured to the changing mat;

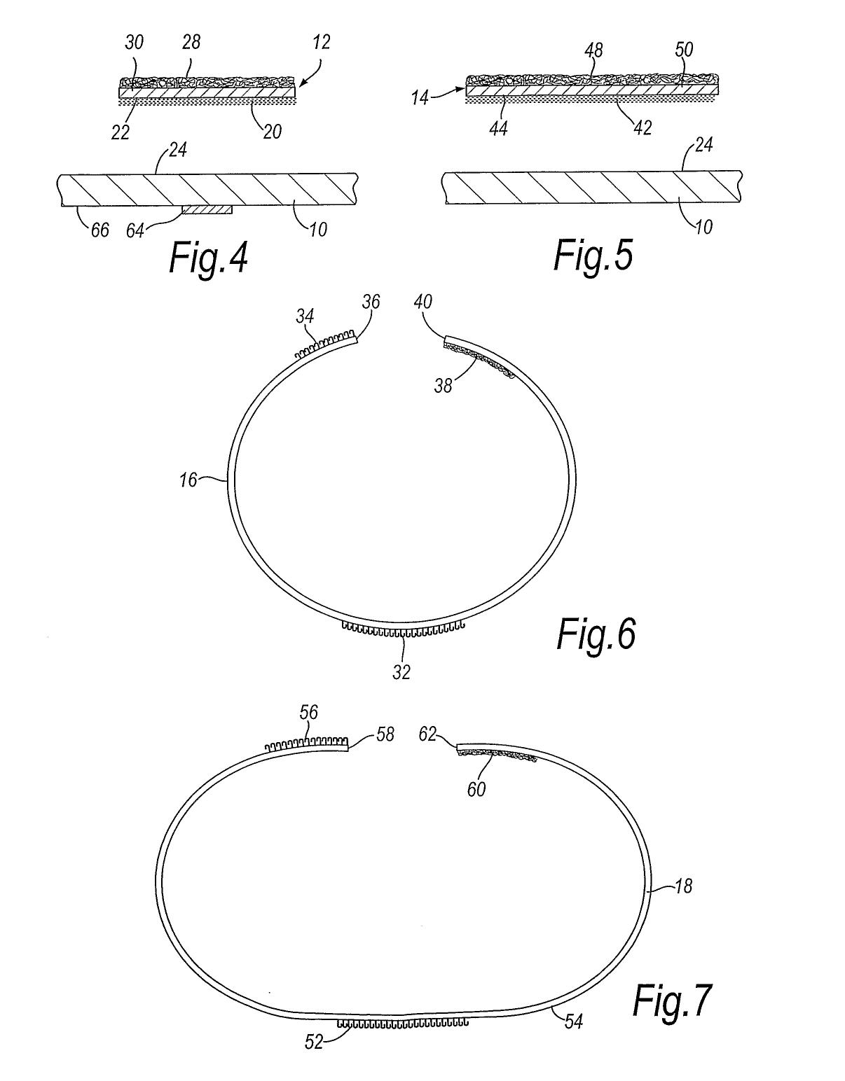

FIG. 5 is a cross-sectional view of the second base member of the kit as it is being secured to the changing mat;

FIG. 6 is a cross-sectional view of one of the upper arm restraints;

FIG. 7 is a cross-sectional view of one of the torso restraint; and

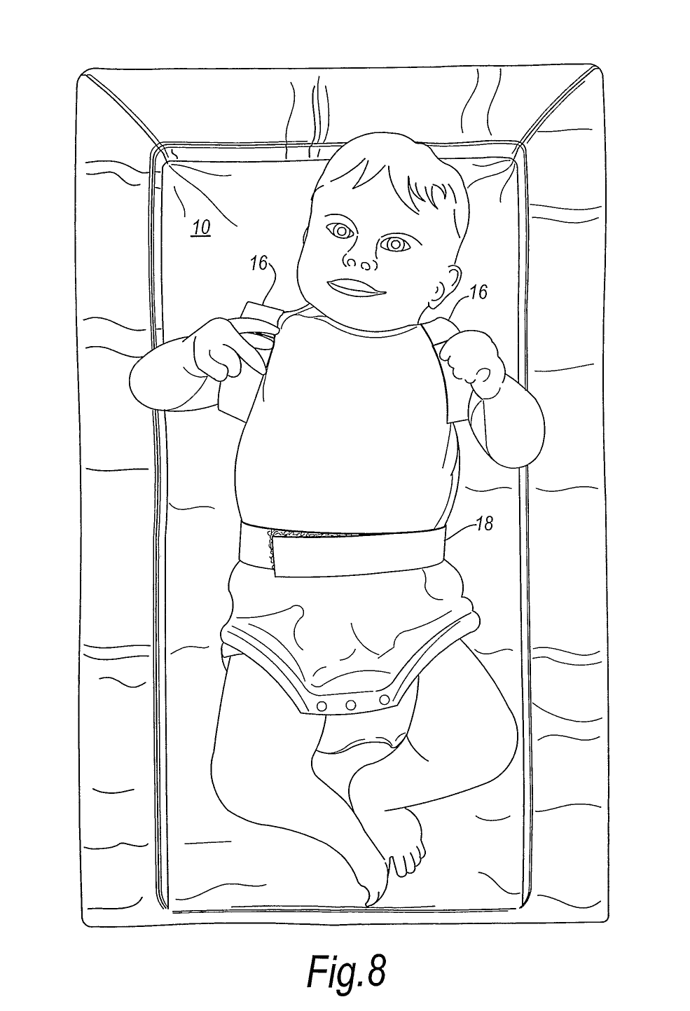

FIG. 8 shows a contented baby restrained by the device formed from the kit.

The drawings show a kit of parts for forming a baby restraining device for use with a baby nappy changing mat 10. The kit includes a first base or support member in the form of an elongate band 12, a separate second base or support member in the form of a rectangular piece 14, left and right upper arm restraint straps 16 and a torso restraint strap 18.

The band 12 has tacky adhesive 20 located on its lower surface 22 for securing it to the upper surface 24 of the mat 10 at a first location 26, to extend in a substantially lateral direction across the mat.

One element 28 of a Velcro™ hook-and-loop system is provided on the upper surface 30 of the band 12 for releasably engaging the upper arm restraint straps 16 selectably in a plurality of laterally disposed positions. The element 28 extends continuously in the lateral direction across the upper surface 30 of the band 12.

Each upper arm restraint strap 16 is formed with the other element 32 of the hook-and-loop system located midway along the strap for releasably engaging the upper arm restraint member 16 with the band 12.

One element 34 of another Velcro™ hook-and-loop system is located on the outside towards one free end 36 of the strap. The other element 38 of the Velcro™ hook-and-loop system is located on the inside towards the other free end 40 of the strap. The elements 34, 38 co-operate to enable the strap to be formed into a loop with adjustable circumference.

The support piece 14 has tacky adhesive 42 located on its lower surface 44 for securing it to the upper surface 24 of the mat 10 at a location 46 spaced from the band 12 and approximately mid-way between the side edges of the mat.

One element 48 of a further Velcro™ hook-and-loop system is formed on the upper surface 50 of the support piece 14 for releasably engaging a torso restraint strap 18, extends continuously in the longitudinal direction along the upper surface 50 of the support piece 14. The other element 52 of Velcro™ hook-and-loop system is formed midway along the outer surface 54 of the torso restraint strap 18.

One element 56 of a still further Velcro™ hook-and-loop system is located on the outside towards one free end 58 of the strap 18. The other element 60 of the Velcro™ system is located on the inside towards the other free end 62 of the strap. The elements 56 and 60 co-operate to enable the strap to be formed into a loop with adjustable circumference.

The kit further includes a stiffening bar 64, to be secured to the lower surface 66 of the changing mat 10, at approximately shoulder level, to further restrain movement of larger and more active babies.

The baby restraining device is constructed by securing the band 12 to the upper surface 24 of a baby nappy changing mat 10 at the first location 26. The upper arm restraint straps 16 are then engaged with the band 12. The support piece 14 is secured to the upper surface 24 of the changing mat 10 at the second location 46. The torso restraint strap 18 is engaged with the second support member 14.

The baby can then be laid onto the mat, with the baby's shoulders against the support band 12 and the baby's lower back against the support piece 14. The upper arm restraint straps 16 are then made into loops around the baby's upper arms, the loops being adjusted to a circumference which is comfortable for the baby. The torso strap 18 is made into a loop around the baby's waist and adjusted to a circumference which is comfortable for the baby. A nappy changing exercise can then proceed.

The upper arm restraint straps 16 can be adjusted laterally, that is towards or away from each other, so as to be positionable at an ideal location, for example to prevent the baby from bringing its hands down to waist level, where it may interfere with the nappy changing exercise.

By the use of orientation-independent engagement between the band 12 and the upper arm restraint straps 16, as is achieved by the use of hook-and-loop fixing systems, it is not essential that the loops formed by the two upper arm restraint straps 16 lie in parallel planes. Indeed, as shown, for maximum comfort, the loops lie in planes which converge towards each other in the direction away from the baby's head.

The torso restraint strap 18 can be adjusted longitudinally, that is towards or away from the upper arm restraint straps 16, so as to be positionable at an ideal location as the baby grows in length.

1. A kit comprising a plurality of parts that form a baby restraining device for use with a changing mat, the kit comprising:
a first base member having a securing element that secures the first base member to an upper surface of the changing mat at a first location and an engagement element on an upper surface of the first base member that releasably engages at least two upper arm restraint members selectably in a plurality of laterally disposed positions;
the at least two upper arm restraint members, wherein the at least two upper arm restraint members are each in the form of a loop having an adjustable circumference and each has on its outer surface an engagement that releasably engages the at least two upper arm restraint members with the first base member;
a second base member having a securing element that secures the second base member to the upper surface of the changing mat at a second location spaced from the first location and an engagement element on an upper surface of the first base member for releasably engaging a torso restraint member; and
the torso restraint member, wherein the torso restraint member is in the form of a loop having an adjustable circumference, the loop having, on its outer surface, an engagement element that releasably engages the torso restraint member with the second base member.

2. The kit of claim 1, wherein the securing element of the first base member is located on a lower surface of the first base member.

3. The kit of claim 1, wherein the first base member is in the form of an elongate band.

4. The kit of claim 1, wherein the securing means of the first base member is in the form of tacky adhesive.

5. The kit of claim 1, wherein the engagement element of the first base member is in the form of one element of a hook-and-loop system, and the engagement means of each of the at least two upper arm restraint members is in the form of another element of the hook-and-loop system.

6. The kit of claim 1, wherein the engagement element of the first base member extends substantially continuously in the lateral direction across the upper surface of the first base member.

7. The kit of claim 1, wherein the at least two upper arm restraint members are each in the form of a strap.

8. The kit of claim 7, wherein the engagement element of each upper arm restraint member is located midway along the strap and co-operating elements are proximal to free ends of the strap to enable the strap to be formed into a loop having an adjustable circumference.

9. The kit of claim 8, wherein the co-operating elements proximal to the free ends of the strap are in the form of a hook-and-loop system, wherein one element of the system is located on the outside of the strap towards one free end thereof and the other element of the system is located on the inside of the strap towards another free end thereof

10. The kit of claim 1, wherein the second base member is non-integral with the first base member.

11. The kit of claim 1, wherein the securing element of the second base member is located on a lower surface of the second base member.

12. The kit of claim 1, wherein the securing element of the second base member is of a tacky adhesive.

13. The kit of claim 1, wherein the engagement element of the second base member is in the form of one element of a hook-and-loop system, while the engagement element of the torso restraint member is in the form of another element of the hook-and-loop system.

14. The kit of claim 1, wherein the engagement element of the second base member extends substantially continuously at least in a longitudinal direction along the upper surface of the second base member.

15. The kit of claim 1, wherein the torso restraint member is in the form of a strap.

16. The kit of claim 15, wherein the engagement element of the torso restraint member is located midway along the strap and co-operating elements are proximal to free ends of the strap to enable the strap to be formed into a loop having an adjustable circumference.

17. The kit of claim 16, wherein the co-operating elements proximal to the free ends of the strap are in the form of a hook-and-loop system, wherein one element of the system is located on the outside of the strap towards one free end thereof and the other element of the system is located on the inside of the strap towards another free end thereof.

18. A baby restraining device comprising:
(i) a changing mat;
(ii) a first base member having a securing element that secures the first base member to an upper surface of the changing mat at a first location and an engagement element on an upper surface of the first base member that releasably engages at least two upper arm restraint members selectably in a plurality of laterally disposed positions;
the at least two upper arm restraint members, wherein the at least two upper arm restraint members are each in the form of a loop having an adjustable circumference and each has on its outer surface an engagement releasably engages the at least two upper arm restraint members with the first base member;
a second base member having a securing element that secures the second base member to the upper surface of the changing mat at a second location spaced from the first location and an engagement element on an upper surface of the first base member for releasably engaging a torso restraint member; and
the torso restraint member, wherein the torso restraint member is in the form of a loop having an adjustable circumference, the loop having, on its outer surface, an engagement element that releasably engages the torso restraint member with the second base member.

19.-34. (canceled)

35. A method of constructing a baby restraining device by use of a kit according to claim 1, including the steps of:
securing the first base member to the upper surface of a baby nappy changing mat at the first location;
engaging the upper arm restraint members with the first base member;
securing the second base member to the upper surface of a changing mat at the second location; and
engaging the torso restraint member with the second base member.

36. A baby restraining device formed by the method of claim 35.
